(19) United States
(12) Patent Application Publication
(10) Pub. No.: US 2012/0051503 A1
(43) Pub. Date: Mar. 1, 2012

(54) STEREOSCOPIC IMAGE DISPLAYING METHOD AND APPARATUS

(75) Inventors: Yasunori OHTA, Kanagawa (JP); Naoyuki Nishino, Kanagawa (JP)

(73) Assignee: FUJIFILM CORPORATION, Tokyo (JP)

(21) Appl. No.: 13/212,957

(22) Filed: Aug. 18, 2011

(30) Foreign Application Priority Data
Aug. 25, 2010 (JP) P2010-188434

(51) Int. Cl. A61B 6/02

(52) U.S. Cl. 378/41

(57) ABSTRACT

A stereoscopic image that is easily viewed by a patient and is capable of reducing the burden on the patient is displayed, and a stereoscopic image for interpretation which includes detailed image information and is viewed by, for example, a doctor is displayed. A radiological image displaying method that radiates radiation to a subject from the two different imaging directions, acquires two radiological images detected by a radiological image detector, and displays a stereoscopic image on a display unit based on the two acquired radiological images, and this method includes: performing image processing on the two radiological images to abstract image information included in the radiological images; and displaying a stereoscopic image based on two abstracted radiological images which are subjected to the image processing and a stereoscopic image based on two radiological images including the image information which is more detailed than that of the abstracted radiological images.

### BACKGROUND OF THE INVENTION

1. Field of the Invention

The present invention relates to a stereoscopic image displaying method and apparatus that radiates radiation to a subject from two different imaging directions, detects two radiological images in the imaging directions, and displays a stereoscopic image based on the two detected radiological images.

2. Description of the Related Art

A technique is known which combines a plurality of images and displays the combined image, thereby enabling stereoscopic viewing using parallax. The image that can be stereoscopically viewed (hereinafter, referred to as a stereoscopic image or a stereo image) is generated based on a plurality of images which are acquired by capturing the same subject in different directions and have parallax therebetween.

Moreover, such way of generating stereoscopic image is utilized not only in the field of digital cameras and televisions but also in the field of capturing a stereoscopic radiological image. That is, a subject is irradiated with radiation from different directions, the radiation passing through the subject is detected by a radiological image detector to acquire plural radiological images having parallax, and a stereoscopic image is generated based on the radiological images. By generating a stereoscopic image in this way, a radiological image with a sense of depth can be observed and thereby more suitable radiological image for diagnosis can be observed.

In some cases, when asking the patient detailed questions of his or her condition in the hospital, the doctor displays a radiological image on the monitor and gives an explanation to the patient while viewing the radiological image together with the patient, considering informed consent.

For example, JP2009-031692A discloses a technique which displays a stereoscopic image viewed by the doctor and a stereoscopic image viewed by the patient on a display, with the doctor and the patient facing each other so as to surround the display. JP2002-091643A discloses a technique which displays a stereoscopic image for a patient.

### SUMMARY OF THE INVENTION

However, as disclosed in JP2009-031692A or JP2002-091643A, when the stereoscopic image is displayed to the patient, it is difficult for the patient who is not used to seeing the stereoscopic images to recognize the stereoscopic image immediately, which may be a burden on the patient.

The present invention has been made in view of the above-mentioned problems and an object of the present invention is to provide a stereoscopic image displaying method and apparatus capable of displaying a stereoscopic image including detailed image information as a stereoscopic image for interpretation to, for example, the doctor and displaying a stereoscopic image that can be easily recognized by the patient and reduce the burden of three-dimensional viewing to the patient.

According to an aspect of the present invention, there is provided a stereoscopic image displaying method that radiates radiation to a subject from the two different imaging directions, detects two radiological images from the two different imaging directions which are captured by the radiating of the radiation using a radiological image detector, and displays a stereoscopic image based on the two detected radiological images. The stereoscopic image displaying method includes: performing image processing on the two radiological images to abstract image information included in the radiological images; and displaying a stereoscopic image based on two abstracted radiological images which are subjected to the image processing and a stereoscopic image based on two radiological images including the image information which is more detailed than that of the abstracted radiological images.

According to another aspect of the present invention, there is provided a stereoscopic image displaying apparatus including: a radiological image acquiring unit that radiates radiation to a subject from the two different imaging directions and acquires radiological images from the two different imaging directions which are captured by the radiating of the radiation and are detected by a radiological image detector; a display unit that displays a stereoscopic image based on the two radiological images acquired by the radiological image acquiring unit; and an image processing unit that performs image processing on the two radiological images to abstract image information included in the radiological images. The display unit displays a first stereoscopic image based on two abstracted radiological images which are processed by the image processing unit and a second stereoscopic image based on two radiological images including the image information which is more detailed than that of the abstracted radiological images.

In the stereoscopic image displaying apparatus according to the above-mentioned aspect, the image processing unit may perform the image processing including gradation processing that makes the contrast of the abstracted radiological image forming the first stereoscopic image more than that of the radiological image forming the second stereoscopic image.

The gradation processing may be two-gradation processing.

The image processing unit may perform the image processing including an edge emphasizing process that emphasizes the edge of the abstracted radiological image forming the first stereoscopic image more than the edge of the radiological image forming the second stereoscopic image.

The image processing unit may perform the image processing including a graininess suppression process that suppresses the graininess of the abstracted radiological image forming the first stereoscopic image than the graininess of the radiological image forming the second stereoscopic image.

The image processing unit may perform the image processing on the radiological image forming the second stereoscopic image, and the image processing unit may perform the image processing and the image abstracting process so as to be switched.

According to the stereoscopic image displaying method and apparatus of the present invention, radiation is radiated to a subject from the two different imaging directions, two radiological images from the two different imaging directions which are detected by a radiological image detector are acquired, and a stereoscopic image is displayed on a display unit based on the two acquired radiological images. Image processing is performed on the two radiological images to abstract image information included in the radiological images, and a stereoscopic image based on two abstracted radiological images which are subjected to the image processing and a stereoscopic image based on two radiological images including the image information which is more detailed than that of the abstracted radiological images are displayed. Therefore, it is possible to display a stereoscopic image including detailed image information as a stereoscopic image for interpretation to, for example, the doctor and display an abstracted stereoscopic image that can be easily recognized by the patient and reduce the burden of stereoscopic viewing to the patient.

### BRIEF DESCRIPTION OF THE DRAWINGS

FIG. 1 is a diagram schematically illustrating the structure of a breast image capture and display system using a stereoscopic image displaying apparatus according to an embodiment of the present invention.

FIG. 2 is a diagram illustrating an arm unit of the breast image capture and display system shown in FIG. 1, as viewed from the right direction of FIG. 1.

FIG. 3 is a block diagram schematically illustrating the internal structure of a computer of the breast image capture and display system shown in FIG. 1.

FIG. 4 is a flowchart illustrating the operation of the breast image capture and display system using the stereoscopic image displaying apparatus according to the embodiment of the present invention.

FIG. 5A is a diagram illustrating an example of a stereo image for interpretation, and FIG. 5B is a diagram illustrating an example of a stereo image for patient observation.

### DESCRIPTION OF THE PREFERRED EMBODIMENTS

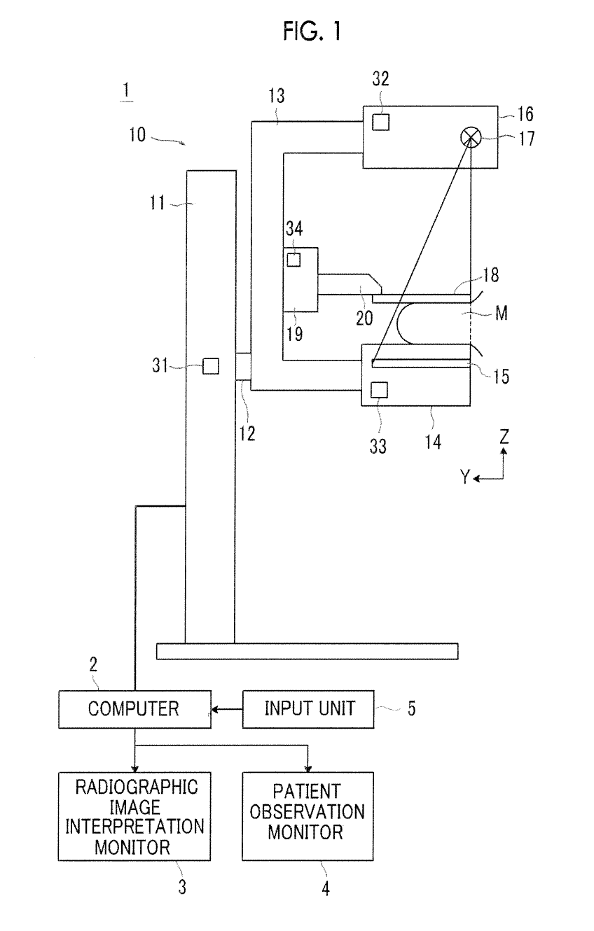

Hereinafter, a breast image capture and display system using a stereoscopic image displayinging apparatus according to an embodiment of the present invention will be described with reference to the accompanying drawings. FIG. 1 is a diagram schematically illustrating the overall structure of the breast image capture and display system according to this embodiment.

As shown in FIG. 1, a breast image capture and display system 1 according to this embodiment includes a breast imaging apparatus 10, a computer 2 that is connected to the breast imaging apparatus 10, and a radiological image interpretation monitor 3 and a patient observation monitor 4 that are connected to the computer 2, and an input unit 5.

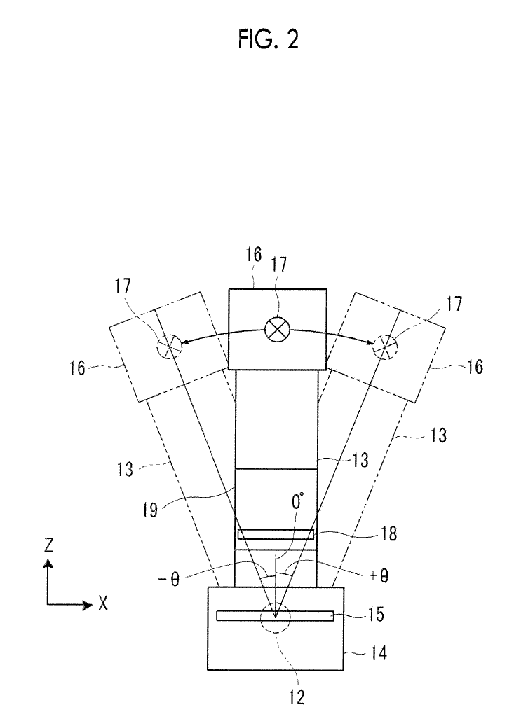

As shown in FIG. 1, the breast imaging apparatus 10 includes a base 11, a rotating shaft 12 that is movable in the vertical direction (Z direction) relative to the base 11 and is rotatable, and an arm unit 13 that is connected to the base 11 by the rotating shaft 12. FIG. 2 shows the arm unit 13, as viewed from the right direction of FIG. 1.

The arm unit 13 has a C-shape and includes one end to which a radiography platform 14 is attached and the other end to which a radiation radiating unit 16 is attached so as to face the radiography platform 14. The rotation and vertical movement of the arm unit 13 are controlled by an arm controller 31 that is incorporated into the base 11.

The radiography platform 14 includes a radiological image detector 15, such as a flat panel detector, and a detector controller 33 that controls, for example, the reading of a charge signal from the radiological image detector 15.

In addition, the radiography platform 14 includes, for example, a circuit board provided with a charge amplifier that converts the charge signal read from the radiological image detector 15 into a voltage signal, a correlated double sampling circuit that samples the voltage signal output from the charge amplifier, and an A/D converter that converts the voltage signal into a digital signal.

The radiography platform 14 is configured so as to be rotatable with respect to the arm unit 13. Therefore, even when the arm unit 13 is rotated with respect to the base 11, the direction of the radiography platform 14 can be fixed with respect to the base 11.

The radiological image detector 15 can repeatedly record and read the radiological image and may be a so-called direct radiological image detector that directly receives radiation and generates charge or a so-called indirect radiological image detector that converts radiation into visible light and then converts the visible light into a charge signal. As a method of reading a radiological image signal, it is preferable to use a so-called TFT reading method of turning on or off a TFT (thin film transistor) switch to read the radiological image signal or a so-called optical reading method of radiating reading light to read the radiological image signal. However, the reading method is not limited thereto, and other methods may be used.

The radiation radiating unit 16 includes a radiation source 17 and a radiation source controller 32. The radiation source controller 32 controls the time when radiation is radiated from the radiation source 17 and the radiation generation conditions (for example, a tube current, time, and a tube current-time product) of the radiation source 17.

In addition, a compression plate 18 that is provided above the radiography platform 14 and compresses the breast, a supporting portion 20 that supports the compression plate 18, and a moving mechanism 19 that moves the supporting portion 20 in the vertical direction (Z direction) are provided at the center of the arm unit 13. The position and compression pressure of the compression plate 18 are controlled by a compression plate controller 34.

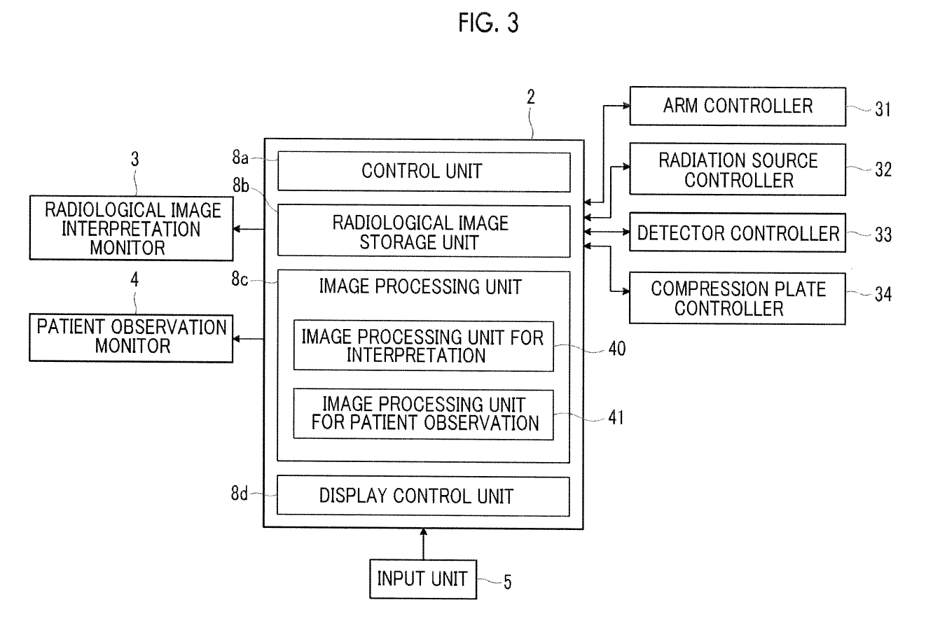

The computer 2 includes, for example, a central processing unit (CPU) and a storage device, such as a semiconductor memory, a hard disk, or an SSD. A control unit 8a. a radiological image storage unit 8b, an image processing unit 8c, and a display control unit 8d shown in FIG. 3 are formed by these hardware components.

The control unit 8a outputs predetermined control signals to various kinds of controllers 31 to 35 to control the entire system. A detailed control method will be described below.

The radiological image storage unit 8b stores the radiological image signals which are generated by an imaging operation from the two different imaging directions and are detected by the radiological image detector 15 in advance.

The image processing unit 8c includes an image processing unit 40 for interpretation and an image processing unit 41 for patient observation.

The image processing unit 40 for interpretation mainly performs image processing on the two radiological image signals stored in the radiological image storage unit 8b such that a radiograph interpreter, such as a doctor, can appropriately interpret the radiological images.

The image processing unit 41 for patient observation performs image processing on the two radiological image signals stored in the radiological image storage unit 8b such that a patient can easily view a stereo image when the patent receives an explanation from the doctor.

The stereo image that is easily viewed by the patient means a stereo image that is easily viewed by the patient who is not used to viewing the stereo images. In this embodiment, in order to generate the stereo image that is easily viewed by/be patient, image processing that abstracts radiological image information is performed on two radiological image signals.

As the image abstracting process, for example, there are following processes: gradation processing of increasing contrast; an edge emphasizing process of emphasizing the edge; and a graininess suppression process of suppressing the graininess of a radiological image.

When the gradation processing is performed in the image abstracting process, specifically, the gradation processing is performed such that the contrast of the radiological image is more than that of the radiological image processed by the image processing unit 40 for interpretation. For example, as the gradation processing, two-gradation processing, that is, so-called binarization may be performed, or δ gradation may be set.

The edge emphasizing process is performed such that the edge of the radiological image is more emphasized than that of the radiological image processed by the image processing unit 40 for interpretation. Examples of the edge emphasizing process include an unsharp mask (USM) process, a high-pass (low-cut) process, and a low resolution (a change from 12 bits to 8 bits).

The graininess suppression process is performed such that the graininess of the radiological image is less than that of the radiological image processed by the image processing unit 40 for interpretation. For example, specifically, a multi-frequency process is used as the graininess suppression process.

The image abstracting process is not limited to the above, but any process may be performed as the image abstracting process as long as it can reduce the burden on the patient of three-dimensional viewing. For example, a process of animating a radiological image or a process of forming an artificial image may be used.

The display control unit 8d performs a predetermined process on the two radiological image signals processed by the image processing unit 40 for interpretation and displays the stereo image of the breast M for interpretation on the radiological image interpretation monitor 3. In addition, the display control unit 8d performs a predetermined process on the two radiological image signals subjected to the image abstracting process by the image processing unit 41 for patient observation and displays the stereo image of the breast M for patient observation on the patient observation monitor 4. Therefore, an image that is more detailed than two radiological images subjected to the image abstracting process is displayed on the radiological image interpretation monitor 3, and an abstracted image subjected to the image abstracting process is displayed on the patient observation monitor 4.

The input unit 5 is, for example, a keyboard or a pointing device, such as a mouse, and receives imaging conditions or an imaging start instruction input from a radiographer.

The radiological image interpretation monitor 3 and the patient observation monitor 4 are configured so as to display a stereo image using two radiological image signals output from the computer 2 when the stereo image is captured. As a structure that displays the stereo image, for example, the following structure may be used in which two radiological images are respectively displayed on two screens based on two radiological image signals and, for example, a half mirror or a polarization glass is used such that one of the two radiological images is incident on the right eye of the observer and the other radiological image is incident on the left eye of the observer, thereby displaying a stereo image. Alternatively, for example, the following structure may be used: a structure in which two radiological images are displayed so as to overlap each other with a positional deviation corresponding to a predetermined amount of parallax therebetween and a polarization glass is used to generate a stereo image such that the observer can view the stereo image; or a structure, such as a parallax barrier type or a lenticular type, in which two radiological images are displayed on a 3D display that can three-dimensionally display the radiological images, thereby generating a stereo image.

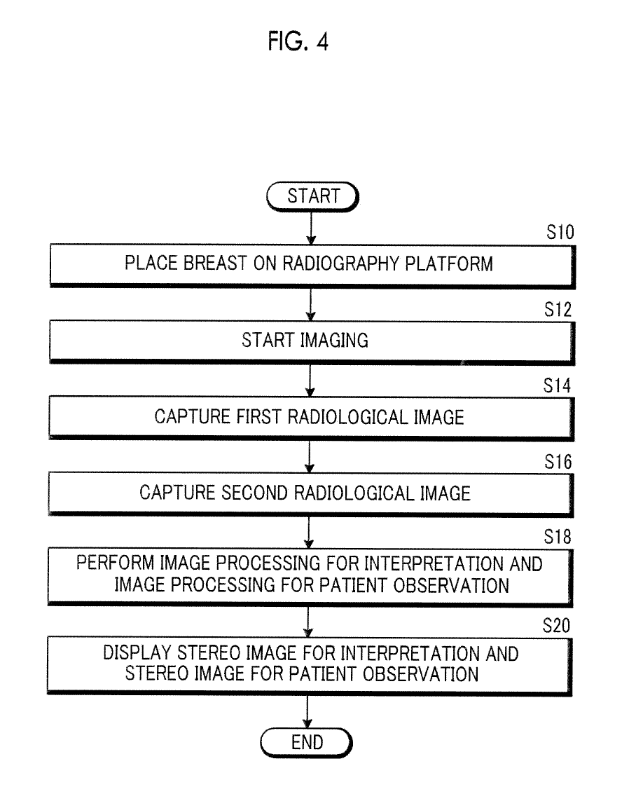

Next, the operation of the breast image capture and display system according to this embodiment will be described with reference to the flowchart shown in FIG. 4.

First, the breast M of the patient is placed on the radiography platform 14 and the compression plate 18 compresses the breast M with a predetermined pressure (S10).

Then, the input unit 5 sequentially receives various kinds of imaging conditions and an image start instruction input from the radiographer (S12).

When the input unit 5 receives the imaging start instruction, a first radiological image of two radiological images forming the stereo image of the breast M is captured (S14).

Specifically, first, the control unit 8a reads the angle of convergence for capturing a predetermined stereo image. In FIG. 2, the angle of convergence is two times more than the absolute value of θ. Then, the control unit 8a outputs the information of the read angle of convergence to/be arm controller 31. In this embodiment, in this case, it is assumed that θ=±2° is stored as the information of the angle of convergence in advance, but the present invention is not limited thereto. The radiographer may use the input unit 5 to set an arbitrary angle of convergence.

The arm controller 31 receives the information of the angle of convergence output from the control unit 8a. Then, the arm controller 31 outputs a control signal based on the information of the angle of convergence such that the arm unit 13 rotates +θ° with respect to the direction vertical to the radiography platform 14, as shown in FIG. 2. That is, in this embodiment, the arm controller 31 outputs a control signal such that the arm unit 13 rotates +2° with respect to the direction vertical to the radiography platform 14.

Then, the arm unit 13 rotates +2° in response to the control signal output from the arm controller 31. In this state, the control unit 8a outputs control signals to the radiation source controller 32 and the detector controller 33 so as to perform the radiating of radiation and the reading of the radiological image signal, respectively. In response to the control signals, the radiation source 17 emits radiation, the radiological image detector 15 detects the radiological image of the breast captured in a +2° direction, and the detector controller 33 reads the radiological image signal. Then, predetermined signal processing is performed on the radiological image signal, and the radiological image signal is stored in the radiological image storage unit 8b of the computer 2.

Then, a second radiological image of the two radiological images forming the stereo image of the breast M is captured (S16).

Specifically, the arm controller 31 outputs a control signal such that the arm unit 13 rotates −θ° with respect to the direction vertical to the radiography platform 14, as shown in FIG. 2. That is, in this embodiment, the arm controller 31 outputs a control signal such that the arm unit 13 rotates −2° with respect to the direction vertical to the radiography platform 14.

Then, the arm unit 13 rotates −2° in response to the control signal output from the arm controller 31. In this state, the control unit 8a outputs control signals to the radiation source controller 32 and the detector controller 33 so as to perform the radiating of radiation and the reading of the radiological image signal, respectively. In response to the control signals, the radiation source 17 emits radiation, the radiological image detector 15 detects the radiological image of the breast captured in a −2° direction, and the detector controller 33 reads the radiological image signal. Then, predetermined signal processing is performed on the radiological image signal, and the radiological image signal is stored in the radiological image storage unit 8b of the computer 2.

Then, the two radiological image signals stored in the radiological image storage unit 8b are input to each of the image processing unit 40 for interpretation and the image processing unit 41 for patient observation. The image processing unit 40 for interpretation performs the above-mentioned predetermined image processing on the two radiological image signals and the image processing unit 41 for patient observation performs the image abstracting process on the two radiological image signals (S18).

Then, the two radiological image signals subjected to image processing by the image processing unit 40 for interpretation are input to the display control unit 8d. Then, a predetermined process is performed on the signals and the processed signals are output to the radiological image interpretation monitor 3. The stereo image of the breast M for interpretation is displayed on the radiological image interpretation monitor 3. The two radiological image signals subjected to the image abstracting process by the image processing unit 41 for patient observation are input to the display control unit 8d. Then, a predetermined process is performed on the signals and the processed signals are output to the patient observation monitor 4. The stereo image of the breast M for patient observation is displayed on the patient observation monitor 4 (S20).

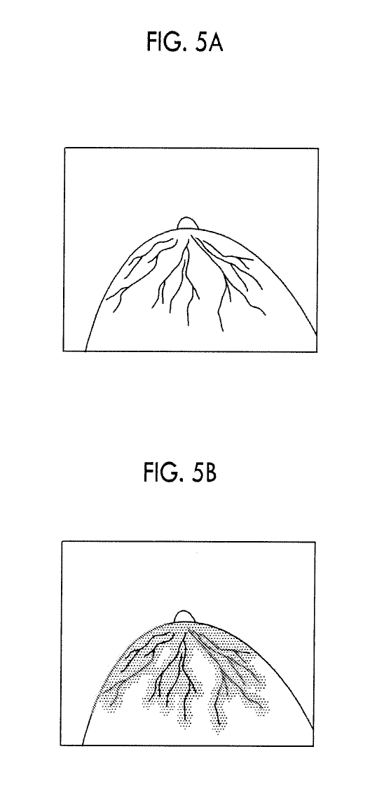

FIG. 5A is a diagram illustrating a stereo image for patient observation displayed on the patient observation monitor 4 and FIG. 5B is a diagram illustrating a stereo image for interpretation displayed on the radiological image interpretation monitor 3.

According to the breast image capture and display system of the above-described embodiment, an abstracted stereo image that is different from a stereo image for interpretation is displayed on the patient observation monitor 4 viewed by the patient. Therefore, it is possible to display the stereo image that is easily viewed by the patient and reduce the burden on the patient when the patient views the stereo image. In addition, a stereo image including detailed image information is displayed on the radiological image interpretation monitor 3 that is viewed by the doctor. Therefore, the doctor can make an appropriate image diagnosis.

In the breast image capture and display system according to the above-described embodiment, the stereo image for patient observation and the stereo image for interpretation are displayed on different monitors. However, the present invention is not limited thereto. The image processing unit 8c may perform switching between image processing for patient observation and image processing for interpretation and the stereo image for patient observation and the stereo image for interpretation may be displayed on one monitor so as to be switched.

As in the above-described embodiment, when the stereo image for patient observation and the stereo image for interpretation are displayed on different monitors, both the stereo image for interpretation and the stereo image for patient observation may be displayed on the radiological image interpretation monitor 3. In this case, for example, the doctor can know the stereo image viewed by the patient and give the patient a smooth explanation.

In the above description, the stereo image displaying apparatus according to an embodiment of the present invention is applied to the breast image capture and display system, but the subject of the present invention is not limited to the breast. For example, the present invention can be applied to a radiological image capture and display system that captures an image of the chest or the head.

What is claimed is:

1. A stereoscopic image displaying apparatus comprising:
a radiation radiating unit that radiates to a subject from two different radiographing directions;
a radiological image detector that detects radiological images from the two different imaging directions which are captured by the radiating of radiation by the radiation radiating unit;
a radiological image acquiring unit that acquires the radiological images from the two different imaging directions detected by the radiological image detector;
a display unit that displays a stereoscopic image based on the two radiological images acquired by the radiological image acquiring unit; and
an image processing unit that performs image processing on the two radiological images to abstract image information included in the radiological images,
wherein the display unit displays a first stereoscopic image based on two abstracted radiological images processed by the image processing unit and a second stereoscopic image based on two radiological images including the image information which is more detailed than that of the abstracted radiological images.

2. The stereoscopic image displaying apparatus according to claim
wherein the image processing unit performs the image processing including gradation processing that makes the contrast of the abstracted radiological image forming the first stereoscopic image more than that of the radiological image forming the second stereoscopic image.

3. The stereoscopic image displaying apparatus according to claim 2,
wherein the gradation processing is two-gradation processing.

4. The stereoscopic image displaying apparatus according to claim 1,
wherein the image processing unit performs the image processing including an edge emphasizing process that emphasizes the edge of the abstracted radiological image forming the first stereoscopic image more than the edge of the radiological image forming the second stereoscopic image.

5. The stereoscopic image displaying apparatus according to claim 2,
wherein the image processing unit performs the image processing including an edge emphasizing process that emphasizes the edge of the abstracted radiological image forming the first stereoscopic image more than the edge of the radiological image forming the second stereoscopic image.

6. The stereoscopic image displaying apparatus according to claim 3,
wherein the image processing unit performs the image processing including an edge emphasizing process that emphasizes the edge of the abstracted radiological image forming the first stereoscopic image more than the edge of the radiological image forming the second stereoscopic image.

7. The stereoscopic image displaying apparatus according to claim 1,
wherein the image processing unit performs the image processing including a graininess suppression process that suppress the graininess of the abstracted radiological image forming the first stereoscopic image than the graininess of the radiological image forming the second stereoscopic image.

8. The stereoscopic image displaying apparatus according to claim 2,
wherein the image processing unit performs the image processing including a graininess suppression process that suppress the graininess of the abstracted radiological image forming the first stereoscopic image than the graininess of the radiological image forming the second stereoscopic image.

9. The stereoscopic image displaying apparatus according to claim 3,
wherein the image processing unit performs the image processing including a graininess suppression process that suppress the graininess of the abstracted radiological image forming the first stereoscopic image than the graininess of the radiological image forming the second stereoscopic image.

10. The stereoscopic image displaying apparatus according to claim 4,
wherein the image processing unit performs the image processing including a graininess suppression process that suppress the graininess of the abstracted radiological image forming the first stereoscopic image than the graininess of the radiological image forming the second stereoscopic image.

11. The stereoscopic image displaying apparatus according to claim 5,
wherein the image processing unit performs the image processing including a graininess suppression process that suppress the graininess of the abstracted radiological image forming the first stereoscopic image than the graininess of the radiological image forming the second stereoscopic image.

12. The stereoscopic image displaying apparatus according to claim 6,
wherein the image processing unit performs the image processing including a graininess suppression process that suppress the graininess of the abstracted radiological image forming the first stereoscopic image than the graininess of the radiological image forming the second stereoscopic image.

13. The stereoscopic image displaying apparatus according to claim 1,
wherein the image processing unit performs the image processing on the radiological image forming the second stereoscopic image, and
the image processing unit performs the image processing and the image abstracting process so as to be switched.

14. The stereoscopic image displaying apparatus according to claim 2,
wherein the image processing unit performs the image processing on the radiological image forming the second stereoscopic image, and
the image processing unit performs the image processing and the image abstracting process so as to be switched.

15. The stereoscopic image displaying apparatus according to claim 3,
wherein the image processing unit performs the image processing on the radiological image forming the second stereoscopic image, and
the image processing unit performs the image processing and the image abstracting process so as to be switched.

16. The stereoscopic image displaying apparatus according to claim 4,
wherein the image processing unit performs the image processing on the radiological image forming the second stereoscopic image, and
the image processing unit performs the image processing and the image abstracting process so as to be switched.

17. The stereoscopic image displaying apparatus according to claim 5,
wherein the image processing unit performs the image processing on the radiological image forming the second stereoscopic image, and
the image processing unit performs the image processing and the image abstracting process so as to be switched.

18. The stereoscopic image displaying apparatus according to claim 6,
wherein the image processing unit performs the image processing on the radiological image forming the second stereoscopic image, and
the image processing unit performs the image processing and the image abstracting process so as to be switched.

19. The stereoscopic image displaying apparatus according to claim 7,
wherein the image processing unit performs the image processing on the radiological image forming the second stereoscopic image, and
the image processing unit performs the image processing and the image abstracting process so as to be switched.

20. A stereoscopic image displaying method that radiates radiation to a subject from the two different imaging directions, detects two radiological images from the two different imaging directions acquired by the radiating of the radiation using a radiological image detector, and displays a stereoscopic image based on the two detected radiological images, comprising:
performing image processing on the two radiological images to abstract image information included in the radiological images; and
displaying a stereoscopic image based on two abstracted radiological images subjected by the image processing and a stereoscopic image based on two radiological images including the image information which is more detailed than that of the abstracted radiological images.
